(19) United States
(12) Patent Application Publication
(10) Pub. No.: US 2026/0171189 A1
(43) Pub. Date: Jun. 18, 2026

(54) SYSTEMS AND METHODS FOR GENERATING SCREENING MAPS USING INTRON-TARGETED CONTROLS

(71) Applicant: RECURSION PHARMACEUTICALS INC., Salt Lake City, UT (US)

(72) Inventors: Marta Marie Fay, Salt Lake City, UT (US); Berton Allen Earnshaw, Cedar Hills, UT (US); Mason Lemoyne Victors, Riverton, UT (US); Lina Maria Nilsson, Salt Lake City, UT (US); August Orvis Allen, Boulder, CO (US); Timothy John Dahlem, Richmond, VA (US); Daniel James Anderson, Redwood City, CA (US); James Douglas Jensen, Farmington, UT (US); Peter Foster McLean, Centerville, UT (US); James Benjamin Taylor, Midlothian, VA (US); Nathan Henry Lazar, Toronto (CA); Safiye Celik, Nashua, NH (US); Conor Austin Forsman Tilllinghast, Salt Lake City, UT (US); Jonathan Curtis Irish, Millcreek, UT (US); Jairus Bradley Pace, Salem, UT (US); Ian Kirk Quigley, Salt Lake City, UT (US); Nicasia Joanne Beebe-Wang, Brooklyn, NY (US); Ryan Christopher Mccomb, Milpitas, CA (US); Jacob Carter Cooper, Sandy, UT (US); Daniel Christian Collinson, West Chester, PA (US); Seyhmus Guler, Salt Lake City, UT (US)

(21) Appl. No.: 19/409,465

(22) Filed: Dec. 4, 2025

Related U.S. Application Data
(60) Provisional application No. 63/733,236, filed on Dec. 12, 2024.

(51) Int. Cl. G16B 20/50; G16B 30/20; G16B 45/00

(52) U.S. Cl. CPC G16B 20/50; G16B 30/20; G16B 45/00

(57) ABSTRACT

A system may include an alignment algorithm and a sample deep learning model configured to output screening maps. The system may also include executable computing instructions that cause one or more processors to receive a plurality of experiment datasets comprising respective unaligned sample perturbation readouts from samples transfected with CRISPR-Cas9 reagents and respective control readouts from control samples transfected with intron targeting control reagents. The one or more processors may also identify intron feature characteristics for the control readouts using the alignment algorithm and generate aligned sample perturbation readouts from the unaligned sample perturbation readouts using the alignment algorithm and the intron feature characteristics identified. The one or more processors may also generate a screening map for the plurality of experiment datasets centered around the control readouts by processing the aligned sample perturbation readouts through the sample deep learning model.

### CROSS REFERENCE TO RELATED APPLICATIONS

This application claims the benefit of U.S. Provisional Application No. 63/733,236 (filed on Dec. 12, 2024), which is incorporated by reference herein in its entirety.

### FIELD

The present disclosure generally relates to systems for generating screening maps of biological samples for use in inferential drug discovery and development, and, more particularly, to systems and methods for generating screening maps using intron targeted controls.

### BACKGROUND

Current sample analysis systems and methods use wild-type cells, mock transfected cell, or cells transfected with non-targeting guide RNAs (e.g., RNAs that do not bind anywhere in the human genome) as controls for single experiments, arrayed whole genome CRISPR screening experiments, or pooled whole genome CRISPR screening experiments. This standard approach is used in both academic and industrial settings. However, none of these approaches mimic the full biological effect of perturbing cells with CRISPR. Specifically, these approaches do not mimic the effect of DNA damage and the cell's biological response to that damage when generating screening maps for these experiments. Other experiment designs for generating screening maps have utilized guides targeting intergenic regions. However, these other designs are also not able to achieve sufficient results because of requirements associated with pulling together the right sets of equipment for the experiments.

As such there is a need for improved control strategies and experiments that are able to accommodate sufficient randomization to produce screening maps capable of mimicking the full biological effect of perturbing cells with CRISPR.

### SUMMARY

In some aspects, the techniques described herein relate to a system for generating screening maps, the system including: one or more processors; a computer memory communicatively coupled to the one or more processors; an alignment algorithm including computing instructions configured for execution by the one or more processors; a sample deep learning model configured to output screening maps; and computing instructions stored on the computer memory, and that when executed by the one or more processors, cause the one or more processors to: receive a plurality of experiment datasets, each of the plurality of experiment datasets including respective unaligned sample perturbation readouts from samples transfected with CRISPR-Cas9 reagents and respective control readouts from control samples transfected with intron targeting control reagents, identify respective intron feature characteristics for the respective control readouts from each of the plurality of experiment datasets using the alignment algorithm, generate aligned sample perturbation readouts from the respective unaligned sample perturbation readouts of each of the plurality of experiment datasets using the alignment algorithm and the respective intron feature characteristics identified for the respective control readouts from each of the plurality of experiment datasets, and generate a screening map for the plurality of experiment datasets centered around the control readouts by processing the aligned sample perturbation readouts through the sample deep learning model.

In some aspects, the techniques described herein relate to a computer implemented method for generating screening maps, the method including: receiving a plurality of experiment datasets, each of the plurality of experiment datasets including respective unaligned sample perturbation readouts from samples transfected with CRISPR-Cas9 reagents and respective control readouts from control samples transfected with intron targeting control reagents; identifying respective intron feature characteristics for the respective control readouts from each of the plurality of experiment datasets using an alignment algorithm; generating aligned sample perturbation readouts from the respective unaligned sample perturbation readouts of each of the plurality of experiment datasets using the alignment algorithm and the respective intron feature characteristics identified for the respective control readouts from each of the plurality of experiment datasets; and generating a screening map for the plurality of experiment datasets centered around the control readouts by processing the aligned sample perturbation readouts through a sample deep learning model that is configured to output screening maps.

### BRIEF DESCRIPTION OF THE DRAWINGS

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1 illustrates a block diagram of a system for generating screening maps using intron targeted controls, in accordance with various embodiments disclosed herein.

FIG. 2 illustrates an example visualization of a screening map generated using the system of FIG. 1, in accordance with various embodiments disclosed herein.

FIG. 3 is a flow diagram of an alignment algorithm, in accordance with various embodiments disclosed herein.

FIG. 4 is a flow diagram of another alignment algorithm, in accordance with various embodiments disclosed herein.

FIG. 5 is a flow diagram of a method for generating screening maps using intron targeted controls, in accordance with various embodiments disclosed herein.

The Figures depict preferred embodiments for purposes of illustration only. Alternative embodiments of the systems and methods illustrated herein may be employed without departing from the principles of the invention described herein.

### DETAILED DESCRIPTION

The systems and methods described herein relate to novel experiment control strategies and related data processing to produce screening maps of biological samples for use in inferential drug discovery and development. In particular, the systems and methods described herein use guide RNAs that target introns of expressed or unexpressed genes and that mimic at least some aspects of targeting exons of expressed or unexpressed genes except for the loss of function of the targeted gene. This approach uniquely allows a combination of the intron guides and experiment reagents to mimic the background effects of double stranded DNA breaks and repair in a cell without affecting transcription or translation. In particular, the intron guides serve as negative controls that are subjected to all of the same experimental and methodological artifacts as everything other cell in an experiment However, the intron guides lack the target gene knockout of the query and positive control agents, which enables the intron guides to manifest the effects of cell inhibition from the transaction media, DNA damage local to the intended cut site, all of the DNA damage response effects, etc. The total set of experiment data may then be normalized to the intron-based control guides. In particular, a multi-dimensional analysis space is may be used for comparisons of experiments from different modalities by aligning the experiment data within the embedding space based on data for control cells applied with the intron guide RNA.

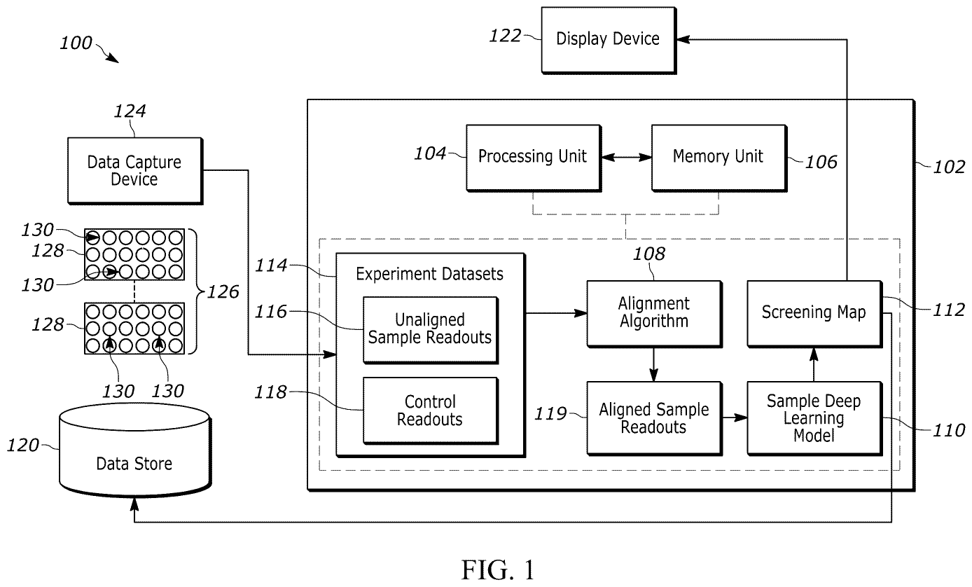

With reference now to FIG. 1, a system 100 for generating for generating screening maps using intron targeted controls is shown. The system 100 includes a computing system 102 such as a local server, remote cloud server, computer, tablet, etc. The computing system 102 may include a processing unit 104 and a memory unit 106.

Processing unit 104 includes one or more processors, each of which may be a programmable microprocessor or the like that executes software or other computing instructions stored in memory unit 106 to execute some or all of the functions of the system 100 as described herein. Processing unit 104 may include one or more graphics processing units (GPUs) and/or one or more central processing units (CPUs), for example. Alternatively, or in addition, one or more processors in processing unit 104 may be other types of processors (e.g., application-specific integrated circuits (ASICs), field-programmable gate arrays (FPGAs), etc.), and some of the functionality of the system 100 as described herein may instead be implemented in hardware.

Memory unit 106 may include one or more volatile and/or non-volatile memories. Any suitable memory type or types may be included in memory unit 106, such as read-only memory (ROM) and/or random access memory (RAM), flash memory, a solid-state drive (SSD), a hard disk drive (HDD), and so on. Collectively, memory unit 106 may store one or more software applications, the data received/used by those applications, and the data output/generated by those applications.

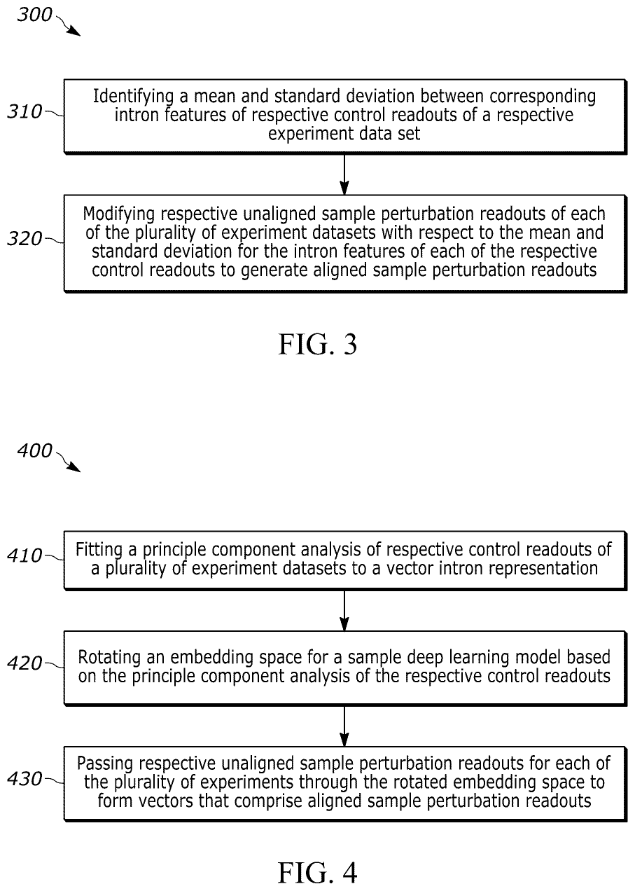

In particular, the memory unit 106 may store instructions for executing an alignment algorithm 108 and a sample deep learning model 110 to generate a screening map 112 from experiment datasets 114 that are input into the alignment algorithm 108 before processing by the deep learning model 110. As shown in FIG. 1, the experiment datasets 114 include unaligned sample readouts 116 and control readouts 118. In general operation, the alignment algorithm 108 is configured to process the unaligned sample readouts 116 relative to the control readouts 118 to generate aligned sample readouts 119. The alignment algorithm 108 may generate the aligned sample readouts 119 by first identifying intron feature characteristics for the control readouts 118 and then modifying the unaligned sample readouts 116 into the aligned sample readouts 119 based on the identified intro feature characteristics. Additional details of the alignment algorithm 108 are described herein in connection with FIGS. 3 and 4. In some embodiments, the processing unit 104 may update parameters of the alignment algorithm 108 on the identified intron feature characteristics for the control readouts 118 and process future unaligned sample perturbation readouts with the alignment algorithm 108 as updated.

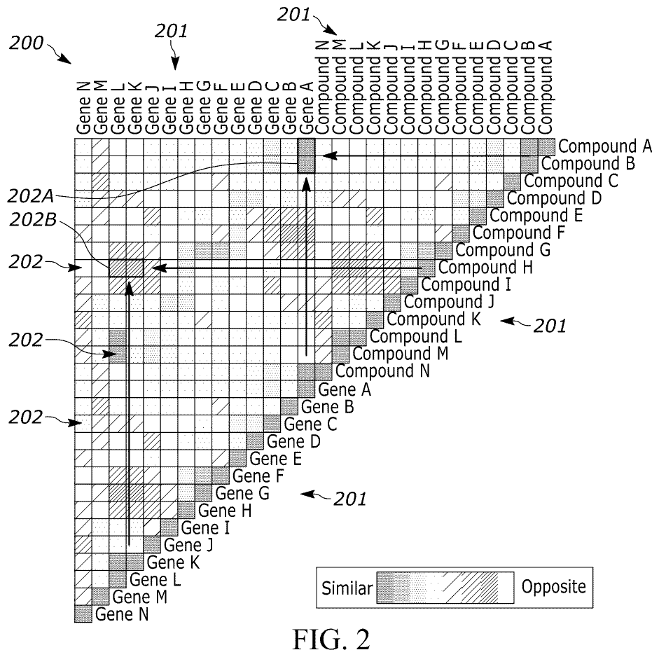

Once the alignment algorithm 108 generates the aligned sample readouts 119, the deep learning model 110 processes the aligned sample readouts 119 such that the screening map 112 is centered around the control readouts 118. For example, the control readouts 118 may be used to define a center or origin point of a multi-dimensional analysis space used by the deep learning model 110 as described in more detail below. Once generated, the screening map 112 may be saved in a data store 120 and/or presented on a display device 122 for use performing inferential drug discovery and development such as by identifying a generic CRISPR-Cas9 transfection cutting phenotype. Additional details of the screening map 112 are described herein in connection with FIG. 2. It should be appreciated that the computing system 102 may employ other models or processes beside the deep learning model 110 to generate the screening map 112. For example, an algorithmic cell profiling process may be used to generate the screening map 112 from the aligned sample readouts 119.

The data store 120 may be implemented as a database, data lake, memory, or other digital storage medium known in the art. Accordingly, the data store 120 may be file system data store, an object-based data store, or other type of data store utilized in the art. Depending on the embodiment, the data store 120 may be implemented locally at the computing system 102, externally at an external data storage service, or a combination thereof. The computing system 102, via the processing unit 104, may be in wired or wireless communication with the external data storage service.

The display device 122 may include a computer monitor or similar graphical display system known in the art that is operably connected to the computing system 102 via wired or wireless means. In some embodiments, the display device 122 may be part of a client or user device (e.g., a personal computer, mobile phone, tablet, etc.). The client device may be operatively coupled to the computing system 102 via wired or wireless means known in the art and may include a user interface. For example, the client device may execute a dedicated application, web browser, etc. as known in the art configured to interface with the computing system 102. In response to user interactions with the computing system 102, the processing unit 104 may provide the screening map 112 and/or other data associated with the computing system 102 to the client device for presentation on the display device 122.

In some embodiments, individual experiment datasets of the experiment datasets 114 may include a respective subset of the unaligned sample readouts 116 and the control readouts 118. Furthermore, the respective subset of the unaligned sample readouts 116 and the control readouts 118 for each individual experiment dataset of the experiment datasets 114 may be data captured by a data capture device 124 from experiments 126 that represent the state of sample material after experimental perturbations are applied to at least a portion of the sample material. For example, each experiment 126 may include a well plate 128 having individual wells 130 populated with experiment samples and control samples. In some embodiments, the experiment samples may include cells, proteins, genes, or other biological material that are transfected with CRISPR-Cas9 reagents and the control samples may include cells, proteins, genes, or other biological material transfected with intron targeting control reagents. The experiment samples may include multiple perturbations (e.g., CRISPR-Cas9 knockout of two genes in a single individual well 130, compound or soluble factor with antisense oligos, etc.). Additionally, experiment samples may include non-CRISPR-Cas9 perturbations such as lentiviral overexpression, and antisense oligos targeting knockdown, and/or other cellular perturbation methods known in the art. In some embodiments at least some of the experiment samples may include cells, proteins, genes, or other biological material that has not been perturbed or mock perturbed.

The intron targeting control reagents may include a set of guide RNAs that target the introns of expressed genes in the control samples as a way to mimic and subtract background signals in transfection-CRISPR-Cas9 knockout screening experiments. The intron guides may include non-naturally occurring RNAs based on naturally occurring genetic information from the human genome. The control samples that are targeted with the intron targeting control reagents may be used as negative controls to mimic the consequences of transfection of CRISPR reagents and the subsequent double strand DNA breaks induced via CRISPR. Furthermore, intron feature characteristics that the alignment algorithm 108 identifies from the control readouts 118 include the feature characteristics of the introns identified in the control samples after being targeted with the intron targeting control reagents.

The intron-based control strategy may be used in piloting and scaling of transfection-based phenomic screening experiments and drug rescue experiments. The intron-based control strategy may also be used in other omics based screening and data production experiments including but not limited to: Phenomics phenoscreens, Phenomics PhenoRescue screens, Phenomics Phenomimics screens, PhenoMap building screens, Secondary confirmation screens, transcriptomics including Trekseq, perturbseq, proteomics, DrugSeq, and more. This control strategy may also be used in some orthogonal validation assays to confirm platform insights/inferences. Furthermore, the intron controls may be used for batch correction and bringing together experiments from different modalities leveraged to perturb the biology of cells as described herein. For example, the intron control strategy may enable the screening map 112 to combine results from CRISPR knockout experiments and compound profiling experiments.

In some embodiments each well plate 128 may include 300-2000 individual wells 130. The control samples may be located in 18-100 of the individual wells 130 and preferably in up to 45 of the individual wells 130. The reaming ones of the individual wells 130 on each well plate 128 may be populated with the experiment samples. In some embodiments, the locations of individual wells 130 containing the control samples and the experiment samples on each well plate 128 is partially or fully randomized. This randomization provides confidence that results shown in the screening map 112 are not effects based on location of a sample within the well plate 128 and allows observations to be based on the underlying biology of the perturbation. In addition to randomizing, each of the experiments 126 may include only a single replicate experiment sample on each well plate 128. This allows for representation of each perturbation in the control or experiment samples to take into account the diversity within the experiments 126 and limit observation of plate-based biased effects in the screening map 112. The combination of randomization with the intron-based control strategy provide for improved resulting screening maps 112 as compared with non-random sample distributions and prior control methods such as non-targeting guides.

In some embodiments, the data capture device 124 may include a confocal microscopy imaging system that captures digital image data of each of the individual wells 130 of the well plate 128 for the experiments 126. The data capture device 124 then sends this image data to the computing system 102. The processing unit 104, upon receiving the image data, may convert the image data into the experiment datasets 114. In particular, the processing unit 104 may be configured to convert the confocal microscopy image data into multi-dimensional vectors that represent the unaligned sample readouts 116 and the screening map 112. It should be appreciated that data capture device 124 may include other devices and methods known in the art that convert biological sample results into machine readable data.

With additional reference now to FIG. 2, the screening map 112 will be discussed in more detail in the context of a heatmap image 200. In general, the screening map 112 output from the deep learning model 110 is data that documents the relationships between different perturbation types 201 applied to the experiment samples of the experiments 126 within the multidimensional analysis space that is centered around the control readouts 118. The perturbation types 201 may include perturbation types of CRISPR-Cas9 gene editing, compounds, soluble factors, etc. The screening map 112 may combine the perturbation types 201 for two or more of the experiments 126 together to enable analysis of information across and within the experiments 126 and to find relationships therebetween. The screening map 112 may combine information for as few as 2-3 experiments 126 and for as many of the experiments 126 needed to cover CRISPR-Cas9 knockdown of all the genes in the genome and over 1 million compounds. The screening map 112 utilizes the introns identified in the control samples in every one of the experiments 126 to define the center of the screening map 112. Utilizing the intron controls allows for several hundred experiments to be added together by combining the different perturbation types into the screening map 112. In particular, the intron control strategy enables all the experiments 126 to be combined into a single multi-dimensional space and to enable improved replication and control for CRISPR-Cas9 genome cutting as compared with prior existing control strategies such as non-targeting guides.

As shown in FIG. 2, the heat map image 200 may include a graphical image or similar representation generated from the screening map 112. In particular, the heat map image 200 may include an arrangement of at least some of the perturbation types 201 with respect to similarity indicators 202 in a grid form as shown in FIG. 2. The similarity indicators 202 may include colors, numbers, or other elements that provide a visual indication of how similar a particular one of the perturbation types 201 is to each of the other perturbation types 201 as indicated by the screening map 112 (e.g., similarity within the multi-dimensional space as centered using the intron control strategy and the alignment algorithm 108 as described herein).

For example, as shown in FIG. 2, the compound A and B perturbation types 201 are similar to the gene A perturbation type 201 as indicated by the similarity indicators 202A and the compound H perturbation type 201 is opposite to the gene L and gene K perturbation types 201 as indicated by the similarity indicators 202B. Analyzing the scope of the similar and opposite indications for the perturbation types 201 can enable inferential drug discovery and development by demonstrating new linkages between different ones of the perturbation types 201.

It should be appreciated that the computing system 102 or a similar system may generate additional visual indicators, text outputs, etc beyond the heat map image 200 using the screening map 112. Furthermore, in some embodiments, the computing system 102 or similar system may generate different heat maps from the screening map 112 that show a particular user defined subset of the perturbation types 201.

With reference now to FIGS. 1, 3, and 4, embodiments of the alignment algorithm 108 shown in FIG. 1 will be discussed in more detail. As described herein, the alignment algorithm 108 uses the representations found in the control readouts 118 of the introns identified in the control samples for each of the experiments 126 to center and align the experiment datasets 114 so that perturbation readouts are placed in a unified and relatable embedding space where the deep learning model 110 constructs the screening map 112. In particular, the alignment algorithm 108 modifies the unaligned sample readouts 116 based on the control readouts 118 to produce the aligned sample readouts 119 which are located in the unified and relatable embedding space. In some embodiments, the aligned sample readouts 119 include multi-dimensional vectors that define a particular point in the multi-dimensional space away from a center or origin defined according to the control readouts 118. In some embodiments, the multi-dimensional space can include 128, 768, 1024, or another number of dimensions and each of the aligned sample readouts 119 may include a vector with different parameters equal to the number of dimensions.

Different embodiments of the alignment algorithm 108 are possible. For example, the alignment algorithm 108 may include a centerscale algorithm 300 shown in FIG. 3 or a typical variation normalization (TVN) algorithm 400 shown in FIG. 4. However, other methods for generating the aligned sample readouts 119 centered in the multi-dimensional space based on the control readouts 118 are also possible.

As shown in FIG. 3, the centerscale algorithm 300, when executed by the processing unit 104 (see FIG. 1), generally relates first order statistics of all the perturbations of a given one of the experiments 126 to the introns identified in the control samples and represented in the control readouts 118.

In particular, at block 310, the centerscale algorithm 300 includes identifying a mean and standard deviation between corresponding intron features of the control readouts 118 of a respective experiment dataset of the experiment datasets 114. In some embodiments, the control readouts 118 may include or be further processed by the processing unit 104 to form a multi-dimensional vector where each element represents a different intron feature. In these embodiments, the mean and standard deviation between the control readouts 118 for each of the experiment datasets 114 is computed individually for all elements in the vectors. In some embodiments, a robust centerscale algorithm may be utilized in place of the centerscale algorithm 300. In these embodiments, a median and median absolute deviation between the corresponding intron features of the control readouts 118 of a respective experiment dataset of the experiment datasets 114 are calculated instead of the mean and standard deviation.

At block 320, the centerscale algorithm 300 includes modifying the unaligned sample perturbation readouts 116 of each of the experiment datasets 114 with respect to the mean and standard deviation for the intron features of each of the respective control readouts 118 to generate the aligned sample perturbation readouts 119. Specifically, multi-dimensional (128, 768, 1024, etc.) vector representations of the unaligned sample readouts 116 for a single experiment dataset 114 are modified to relate to the mean and standard deviation identified from the 128 dimensional vector representations of the control readouts 118 from the same one of the experiment datasets 114. The complete set of the aligned sample readouts 119 that are input into the deep learning model 110 include the completed results of this process for every one of the experiment datasets 114. Furthermore, because each of the experiments 126 include the same set of intron controls, relation of the unaligned sample readouts 116 to the mean and standard deviation of the control readouts 118 associated with the identified introns scales and centers the multi-dimensional dimensional space for all of the aligned sample readouts 119 according to the intron representation.

As shown in FIG. 4, the TVN algorithm 400, when executed by the processing unit 104 (see FIG. 1), generally operates to rotate the embedding space for the deep learning model 110 using principal component analysis of the control readouts 118 and correlation matching or alignment on the second order statistics between the control readouts 118 for the individual wells 130 containing the control samples and the unaligned sample readouts 116 from the other individual wells 130 of the experiments 126. In particular, a correlation alignment (CORAL) method may be used to align the control readouts 118 using second order statistics. For example, CORAL may compute the correlation of the entire dataset, then each of the experiments individually, then remove the experiment-level correlation to apply a whole-dataset correlation. In essence, CORAL is a process by which all of the experiment correlation is normalized to that of the entire dataset. CORAL achieves similar results to center scaling as described except that CORAL which is applied on the correlation matrix and not directly on the data.

In particular, at block 410, the TVN algorithm 400 includes fitting a principal component analysis of the control readouts control readouts 118 of the experiment datasets 114 to a vector intron representation. This fitting of the principal component analysis may identify respective intron feature characteristics for the control readouts 118.

At block 420, the TVN algorithm 400 includes rotating an embedding space for the deep learning model 110 based on the principal component analysis of the control readouts 118.

At block 430, the TVN algorithm 400 includes passing the unaligned sample readouts 116 for each of the experiment datasets 114 through the rotated embedding space to form vectors that comprise the aligned sample readouts 119.

As described above, the computing system 102 may employ alternative methods to generate the aligned sample readouts 119 beyond the centerscale algorithm 300 and the TVN algorithm 400 described above in connection with FIGS. 3 and 4. For example, methods that align means, variance, and covariance distributions in the control readouts 118 and unaligned sample readouts 116; perform linear transformations to reduce the variance that can be attributed to batch variables (e.g. Principal Component Analysis, Canonical Correlation Analysis, Typical Variance Normalization, Procrustes analysis, etc.); and perform non-linear transformations like Variational Autoencoders and other deep learning networks.

Furthermore, some or all of the methods described herein may be assisted by prescaling the intron control cells on each well plate 128 to ensure that the distribution across all plates for the experiments 126 is within the same degree of variance. This prescaling removes variance that exists across the same plates within an experiment. Because the technical perturbations are repeated across multiple well plates 128 and location randomization is used, the intron controls may be used to ensure that the same results are achieved across the well plates 128 and that the normalization across the well plates 128 is not due to position.

Further still, batch correction techniques may be employed to further refine the experiment datasets 114. For example, an implementation of the COMBAT batch-correction method may be used to correct for plate-level batch effects with a unique plate identifier being used as the batch key. COMBAT uses an empirical Bayes approach to model batch effect and adjusts both mean and variance to remove any identified batch effects while preserving relevant biological signals. In more detail, the experiment datasets 114 are first normalized by raw gene counts using median-of-ratios normalization. Then, a log-transformation is applied before implementing the COMBAT batch correction methods. The COMBAT method standard-scales the normalized log-transformed gene counts, fits a linear model to estimate batch and biological variation, calculates empirical Bayes estimates of mean and variance corrections for batch effect across all genes in the experiment datasets 114, and applies these corrections to the experiment datasets 114. Applying COMBAT effectively reduces plate-level batch effect in the experiment datasets 114 for more effective analysis of biological perturbation relationships across the well plates 128.

In some embodiments, the computing system 102 may determine an experiment-level alignment score based on calculation of cosine distances of the intron controls (e.g., the control readouts 118 in a certain experiment with respect to the control readouts 118 for all other experiments. This experiment-level alignment score can be used to monitor changes in alignment quality over time or across experiment types or compound libraries. In particular, the experiment-level alignment score enables comparison of experiments that use different alignment methods described herein or embeddings from different models.

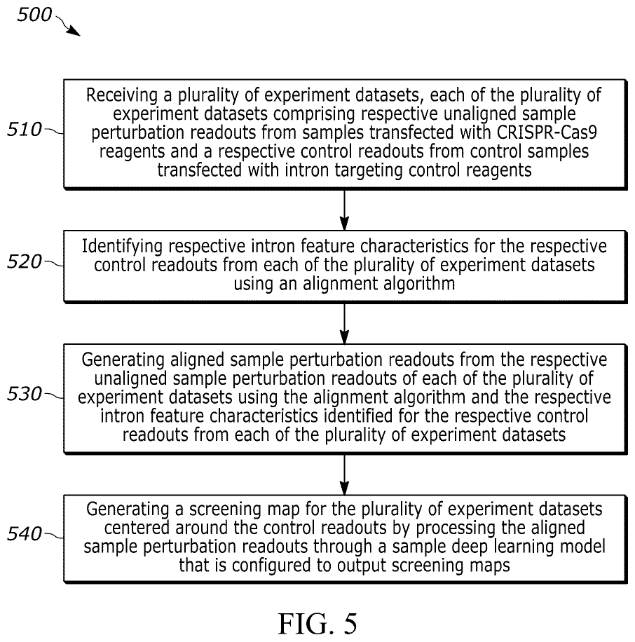

FIG. 5 shows a method 500 for generating the screening map 112 using the alignment algorithm 108 and the deep learning model 110. The method 500 may be executed by the computing system 102 via execution by the processing unit 104 of instructions stored on the memory unit 106.

At block 510, the method 500 includes receiving a plurality of experiment datasets (e.g., the experiment datasets 114). Each of the plurality of experiment datasets include respective unaligned sample perturbation readouts (e.g., the unaligned sample readouts 116) from samples transfected with CRISPR-Cas9 reagents and respective control readouts (e.g., control readouts 118) from control samples transfected with intron targeting control reagents.

At block 520, the method 500 includes identifying respective intron feature characteristics for the respective control readouts from each of the plurality of experiment datasets using an alignment algorithm. (e.g., the alignment algorithm 108). In embodiments where the alignment algorithm includes a centerscale algorithm (e.g., the centerscale algorithm 300), identifying the respective intron feature characteristics for the respective control readouts using the centerscale algorithm includes identifying a mean and standard deviation between corresponding intron features of each of the respective control readouts of the respective experiment dataset. In embodiments where the alignment algorithm includes a variation normalization algorithm (e.g., the TVN algorithm 400), identifying the respective intron feature characteristics for the respective control readouts using the variation normalization algorithm includes fitting a principal component analysis of the respective control readouts to a vector intron representation.

At block 530, the method 500 includes generating aligned sample perturbation readouts (e.g., the aligned sample readouts 119) from the respective unaligned sample perturbation readouts of each of the plurality of experiment datasets using the alignment algorithm and the respective intron feature characteristics identified for the respective control readouts from each of the plurality of experiment datasets. In embodiments where the alignment algorithm includes the centerscale algorithm, generating the aligned sample perturbation readouts using the centerscale algorithm includes modifying the respective unaligned sample perturbation readouts of each of the plurality of experiment datasets with respect to the mean and standard deviation for the intron features of each of the respective control readouts. In embodiments where the alignment algorithm includes the variation normalization algorithm, generating the aligned sample perturbation readouts using the variation normalization algorithm includes rotating an embedding space for the sample deep learning model based on the principal component analysis of the respective control readouts and passing the respective unaligned sample perturbation readouts for each of the plurality of experiments through the rotated embedding space to form vectors that comprise the aligned sample perturbation readouts.

At block 540, the method 500 includes generating a screening map (e.g., screening map 112) for the plurality of experiment datasets centered around the control readouts by processing the aligned sample perturbation readouts through a sample deep learning model that is configured to output screening maps.

In some embodiments, the method 500 may also include updating at least some parameters of the alignment algorithm based on the respective intron feature characteristics for the respective control readouts from each of the plurality of experiment datasets and processing future unaligned sample perturbation readouts with the alignment algorithm as updated. Furthermore, the method 500 may include identifying a generic CRISPR-Cas9 transfection cutting phenotype used for inferential drug discovery and development using the screening map for the plurality of experiment datasets. The method 500 may also include receiving confocal microscopy image data associated with the plurality of experiment datasets (e.g., image data from the data capture device 124) and converting the confocal microscopy image data into multi-dimensional vectors that represent the respective sample perturbation readouts and the respective control readouts. The confocal microscopy image data may be taken form a respective well plate (e.g. well plate 128) for each of the plurality of experiment datasets. The respective well plate for each of the plurality of experiment datasets includes the samples transfected with CRISPR-Cas9 reagents and the control samples transfected with intron targeting control reagents.

Although the disclosure herein sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality and improve the functioning of conventional computers.

What is claimed is:

1. A system for generating screening maps, the system comprising:
one or more processors;
a computer memory communicatively coupled to the one or more processors;
an alignment algorithm comprising computing instructions configured for execution by the one or more processors;
a sample deep learning model configured to output screening maps; and
computing instructions stored on the computer memory, and that when executed by the one or more processors, cause the one or more processors to:
receive a plurality of experiment datasets, each of the plurality of experiment datasets comprising respective unaligned sample perturbation readouts from samples transfected with CRISPR-Cas9 reagents and respective control readouts from control samples transfected with intron targeting control reagents,
identify respective intron feature characteristics for the respective control readouts from each of the plurality of experiment datasets using the alignment algorithm,
generate aligned sample perturbation readouts from the respective unaligned sample perturbation readouts of each of the plurality of experiment datasets using the alignment algorithm and the respective intron feature characteristics identified for the respective control readouts from each of the plurality of experiment datasets, and
generate a screening map for the plurality of experiment datasets centered around the control readouts by processing the aligned sample perturbation readouts through the sample deep learning model.

2. The system of claim 1 wherein the alignment algorithm comprises a centerscale algorithm, wherein the respective intron feature characteristics for the respective control readouts comprise a mean and standard deviation between corresponding intron features of each of the respective control readouts of the respective experiment dataset, and wherein the aligned sample perturbation readouts comprise modifications of the respective unaligned sample perturbation readouts of each of the plurality of experiment datasets with respect to the mean and standard deviation for the intron features of each of the respective control readouts.

3. The system of claim 1 wherein the alignment algorithm comprises a variation normalization algorithm, wherein a principal component analysis of the respective control readouts fit to a vector intron representation comprise the respective intron feature characteristics, wherein the variation normalization algorithm rotates an embedding space for the sample deep learning model based on the principal component analysis of the respective control readouts, and wherein the aligned sample perturbation readouts comprises vectors formed from passing the respective unaligned sample perturbation readouts for each of the plurality of experiments through the rotated embedding space.

4. The system of claim 1 wherein the samples transfected with CRISPR-Cas9 reagents and the control samples transfected with intron targeting control reagents are located on a respective well plate for each of the plurality of experiment datasets.

5. The system of claim 4 wherein the respective well plate comprises 300-2000 individual wells.

6. The system of claim 5 wherein the control samples are located in 18-100 of the individual wells.

7. The system of claim 4 wherein the control samples are located in 45 wells of the respective well plate.

8. The system of claim 4 wherein the instructions further cause the one or more processors to:
receive confocal microscopy image data of the respective well plate for each of the plurality of experiment datasets; and
convert the confocal microscopy image data into multi-dimensional vectors that represent the respective sample perturbation readouts and the respective control readouts.

9. The system of claim 1 wherein the instructions further cause the one or more processors to:
update at least some parameters of the alignment algorithm based on the respective intron feature characteristics for the respective control readouts from each of the plurality of experiment datasets.

10. The system of claim 9 wherein the instructions further cause the one or more processors to:
process future unaligned sample perturbation readouts with the alignment algorithm as updated.

11. The system of claim 1 wherein the instructions further cause the one or more processors to:
generate one or more heatmap images for the plurality of experiment datasets using the screening map, wherein the one or more heatmap images are used to identify a generic CRISPR-Cas9 nucleofection cutting phenotype used for inferential drug discovery and development.

12. The system of claim 1 wherein the instructions further cause the one or more processors to:
receive confocal microscopy image data associated with the plurality of experiment datasets; and
convert the confocal microscopy image data into multi-dimensional vectors that represent the respective sample perturbation readouts and the respective control readouts.

13. A computer implemented method for generating screening maps, the method comprising:
receiving a plurality of experiment datasets, each of the plurality of experiment datasets comprising respective unaligned sample perturbation readouts from samples transfected with CRISPR-Cas9 reagents and respective control readouts from control samples transfected with intron targeting control reagents;
identifying respective intron feature characteristics for the respective control readouts from each of the plurality of experiment datasets using an alignment algorithm;
generating aligned sample perturbation readouts from the respective unaligned sample perturbation readouts of each of the plurality of experiment datasets using the alignment algorithm and the respective intron feature characteristics identified for the respective control readouts from each of the plurality of experiment datasets; and
generating a screening map for the plurality of experiment datasets centered around the control readouts by processing the aligned sample perturbation readouts through a sample deep learning model that is configured to output screening maps.

14. The computer implemented method of claim 13 wherein:
the alignment algorithm comprises a centerscale algorithm;
identifying the respective intron feature characteristics for the respective control readouts using the centerscale algorithm includes identifying a mean and standard deviation between corresponding intron features of each of the respective control readouts of the respective experiment dataset; and
generating the aligned sample perturbation readouts using the centerscale algorithm includes modifying the respective unaligned sample perturbation readouts of each of the plurality of experiment datasets with respect to the mean and standard deviation for the intron features of each of the respective control readouts.

15. The computer implemented method of claim 13 wherein:
the alignment algorithm comprises a variation normalization algorithm;
identifying the respective intron feature characteristics for the respective control readouts using the variation normalization algorithm includes fitting a principal component analysis of the respective control readouts to a vector intron representation; and
generating the aligned sample perturbation readouts using the variation normalization algorithm includes:
rotating an embedding space for the sample deep learning model based on the principal component analysis of the respective control readouts, and
passing the respective unaligned sample perturbation readouts for each of the plurality of experiments through the rotated embedding space to form vectors that comprise the aligned sample perturbation readouts.

16. The computer implemented method of claim 13 further comprising:
updating at least some parameters of the alignment algorithm based on the respective intron feature characteristics for the respective control readouts from each of the plurality of experiment datasets.

17. The computer implemented method of claim 16 further comprising:
processing future unaligned sample perturbation readouts with the alignment algorithm as updated.

18. The computer implemented method of claim 13 further comprising:
generating one or more heatmap images for the plurality of experiment datasets using the screening map; and
identifying a generic CRISPR-Cas9 nucleofection cutting phenotype used for inferential drug discovery and development using the one or more heatmap images.

19. The computer implemented method of claim 13 further comprising:
receiving confocal microscopy image data associated with the plurality of experiment datasets; and
converting the confocal microscopy image data into multi-dimensional vectors that represent the respective sample perturbation readouts and the respective control readouts.

20. The computer implemented method of claim 19 wherein:
the confocal microscopy image data is taken form a respective well plate for each of the plurality of experiment datasets; and
the respective well plate for each of the plurality of experiment datasets includes the samples transfected with CRISPR-Cas9 reagents and the control samples transfected with intron targeting control reagents.
